(19) United States
(12) Patent Application Publication
(10) Pub. No.: US 2011/0054025 A1
(43) Pub. Date: Mar. 3, 2011

(54) USE OF A PENTACYCLIC TRITERPENE IN A PHARMACEUTICAL COMPOSITION FOR THE TREATMENT OF MULTIPLE SCLEROSIS

(75) Inventors: María Luisa Nieto Callejo, Valladolid (ES); Rubén Martín Montaña, Valladolid (ES); Juliana Carvalho Tavares, Valladolid (ES); Valentina Ruiz Gutiérrez, Sevilla (ES)

(73) Assignee: CONSEJO SUPERIOR DE INVESTIGACIONES CIENTIFICAS, Madrid (ES)

(21) Appl. No.: 12/935,233

(22) PCT Filed: Feb. 12, 2009
(86) PCT No.: PCT/ES2009/070024

§ 371 Date: Nov. 18, 2010

(30) Foreign Application Priority Data
Mar. 28, 2008 (ES) P200800873

(51) Int. Cl. A61K 31/19; A61K 31/047; A61P 25/28

(52) U.S. Cl. 514/557; 514/729

(57) ABSTRACT

Oleanolic acid (3β-hiydroxyolean-12-en-28-oic acid) is a pentacyclic triterpene occurring in a large number of medicinal plants. The present invention is directed to the pharmacological application of the oleanolic acid, alone or in combination with other substances, in the treatment or prophylaxis of neurodegenerative diseases such as multiple sclerosis. The inventors have found that oleanolic acid, intraperitoneally administered once daily, reduces significantly the immuno-inflammatory and neurological symptoms associated with the experimental autoimmune encephalomyelitis, an experimental model of multiple sclerosis, delaying the onset and reducing the progression of the disease.

### FIELD OF THE INVENTION

The present invention contemplates a pharmacological composition comprising as an active ingredient oleanolic acid, for the prophylaxis and treatment of neurodegenerative diseases such as multiple sclerosis. The invention demonstrates the ability of this natural compound, present in the cuticle of olives and olive leaves, as well as in oils where these fractions have a significant presence (olive-pomace oil), to reduce markedly the clinical signs of experimental autoimmune encephalomyelitis, referred to herein as EAE. This action is associated with an increase of both weight and survival of experimental animals as well as a reduction of the inflammatory reaction and cerebrovascular permeability. It is therefore an object of this invention to provide a pharmaceutical and/or nutraceutical composition with applications for treating Multiple Sclerosis (hereafter MS).

### BACKGROUND OF THE INVENTION

Multiple Sclerosis is an autoimmune, inflammatory and degenerative disease of the central nervous system (CNS) directed against myelin proteins of the brain and spinal cord. It is considered as one of the major neurological illness of young adults, but although more common in women, the severity of the disease is more pronounced in male carriers. MS is characterized by the presence of plaques or lesions of demyelination in the white matter of the CNS, resulting in an abnormal nerve conduction that mainly affects muscle control. Signs and symptoms vary widely, depending on the location of the lesions. Thus, i) motor symptoms may include speech impairment, weakness, tremors and difficulty walking, ii) sensory symptoms may include numbness, tingling, pain and visual disturbances and iii) the psychological symptoms can include changes in mood, and depression. In most patients, the disease occurs during or after a previous stage of relapses and remissions, while in a small percentage of patients (10-15%), the evolution of the disease is progressive from the beginning. The attacks lead to a continuous process of demyelination and remyelination, which causes scarring of nerve fibbers and a progressive disability. Although the exact cause of the disease is still unknown, several studies have supported the hypothesis of a viral aetiology, but none of the viruses studied has emerged as the causative agent searched. So, current theories suggest that the etiology of MS may be related to a combination of autoimmune, environmental and viral factors that act together with an underlying genetic predisposition.

Experimental autoimmune encephalomyelitis (EAE) induced in susceptible strains of animals provides the best available model for understanding events in MS and to test new drugs that could lead to novel therapies (Raine et al. Lab Invest. 1980, 43: 150-7). In this model, immunization of the CNS is achieved by injection with specific antigens (myelin basic protein, proteolipid protein or myelin/oligodendrocyte glycoprotein) and adjuvants, which induce a T cell-mediated attack on the brain and spinal cord (Pettinelli and McFarlin, J. Immunol. 1981, 127: 1420-1423). Thus, in the CNS of animals with EAE are observed perivascular and parenchymal inflammatory infiltrates consisting mainly of (CD8⁺ and CD4⁺) T cells, B cells and activated macrophages that have crossed the blood-brain barrier (BBB), due to alterations in its permeability. Subsequently, it takes place the activation of resident cells such as microglia and astrocytes (Schonrock L M. et al Neuropathol Appl Neurobiol. 1998, 24: 320-30). Depending on the species and/or animal strain as well as on the antigen used, the inflammatory lesions may be accompanied, or not, by areas of demyelination. One of the characteristic features of EAE is the progressive weight loss during the clinical phase of the disease, which is rapidly reverted when the animals recover (Ruuls et al, J. Immunology, 1996, 157:5721-5731). The recovery is also associated with the production of cytokines such as IL-10 and TGF-β (Kennedy et al., J. Immunol. 1992, 149:2496-2505).

The currently available treatments for multiple sclerosis are intended to suppress the immune-inflammatory component of the disease. At present, the disorder is treated symptomatically by administration of high-dose of glucocorticoids at the onset of acute neurological symptoms. However, due to the numerous and serious side effects of steroids, it is not possible to carry out continuous preventive therapy. Some patients also received immunosuppressive agents, although they are often poorly tolerated. Therefore it is necessary to identify new treatments for MS to resolve the above mentioned issues and to minimize or slow the progression of the disease.

Thus, pharmacological research is focused on finding novel therapeutic agents, and in recent years the plant kingdom is proving to be an excellent resource for finding these new compounds. Even, it have been developed several lines of research that seek to analyze the beneficial effects of natural compounds present in vegetables, in order to relate the composition of the diet with the development/modulation of immune-inflammatory diseases, to define possible nutritional therapeutic strategies. In this context, oleanolic acid is a pentacyclic triterpenoid of oleanane group frequently present in plants used in traditional medicine in different countries. It comes in the form of free acid or as triterpenoid saponins aglycones (Liu J., J Ethnopharmacol 1995, 49: 57-68), and has been isolated from various plant species, including Olea europaea.

The literature since 1906 (Canzoneri F., Gazz chim ital, 1906, 36:372) recognize that the oleanolic acid as a permanent constituent of olive leaves and fruits. In the olive tree, is mainly in leaves, green fruits, especially in the cuticle, and in the residual liquor from the dressing olives. It is also found in mature olives, olive oil and pomace oil.

There are numerous publications on the therapeutic properties of triterpenes and in particular there are multiple studies that collect the biological and pharmacological activities of the oleanolic acid. These include: hepatoprotective (Liu Y et al., Toxicology Letters 1998, 95: 77-85), inflammatory (Mañez S et al. Eur J Pharmacol. 1999, 334: 103-5; Marquez-Martin A et al. Cytokines 2006), antitumoral (R. Martin et al. Cancer Res 2007, 67: 3741-51, John M E et al. J Nutr. 2006, 136: 2553-7), anti-HIV (Zhu Y M et al., Bioorg Med Chem Lett. 2001, 11: 3115-8; Kashiwara Y et al., J Nat Prod 1998, 61: 1090-5), vasodilatory (R. Rodriguez-Rodriguez et al. Br J Nutr. 2004, 92: 635-42), hypoglycaemic (H. Sato et al. Biochem Biophys Res Commun. 2007, 362: 793-8, Yoshikawa M et al. Biofactors. 2000, 13: 231-7), and lipid lowering activities (BL Ma. Traditional Medicine and Pharmacology. 1986, 2: 28-29).

The present invention relates to the search for new treatments for MS and describes a new pharmacological application of the oleanolic acid, as an agent that markedly reduces the clinical and immuno-inflammatory hallmarks of the experimental autoimmune encephalomyelitis.

### SUMMARY OF THE INVENTION

### Brief Description

The present invention describes the use of a pentacyclic triterpene, on the development of drugs or pharmaceutical compositions and nutraceutical compositions for the prevention and/or treatment of neurodegenerative diseases, such as multiple sclerosis. More particularly, the compound of the present disclosure refers to the pentacyclic triterpene, oleanolic acid. (3β-hydroxyolean-12-en-28-oic acid).

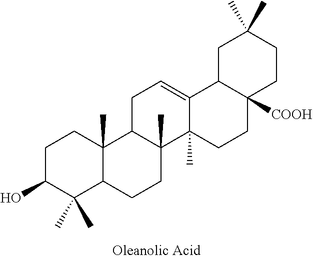

It is also part of the invention a pharmaceutical composition useful for the treatment of a neurodegenerative disease, preferably multiple sclerosis comprising a therapeutically effective amount of a pentacyclic triterpene, together with, optionally, one or more adjuvants and/or pharmaceutically acceptable vehicles.

In addition, a further aspect within the scope of the invention relates to a pharmaceutical composition in which the pentacyclic triterpene is oleanolic acid, and the use of the pharmaceutical composition to treat a human being affected by a neurodegenerative disorder, preferably multiple sclerosis, involving the administration of the therapeutic composition to reduce the progression of the disease.

### DETAILED DESCRIPTION

This invention pertains to the use of a composition comprising oleanolic acid as the agent that reduces the clinical and the immuno-inflammatory signs observed in the experimental autoimmune encephalomyelitis induced in C57BL/6 mice (female 6-8 weeks of age), animal model of multiple sclerosis, and that in addition significantly delays the onset of the disease, in particular (see Example 1):

a) Daily ip administration of 6 mg/kg of oleanolic acid (OA1) decreases the severity of the disease, and

b) Daily ip administration of 6 mg/kg of oleanolic acid (OA2) delays the onset of EAE.

These actions are manifested through: i) amelioration neurological symptoms, ii) weight gain, iii) decrease of cellular and molecular extravasation, iv) decrease expression of key proteins in inflammatory processes, v) increased survival of EAE mice.

Therefore, the results suggest that this triterpene may have protective effects on the BBB integrity and on the inflammatory events related to the development of EAE, which could lead to an improvement of the clinical symptoms associated with MS, in the EAE model, as well as other neurodegenerative disorders. Furthermore, since this product is a natural substance that can be isolated from olives, this compound could be used as a supplement to the diet or in nutraceutical preparations.

Thus, an object of this invention is the use of a pentacyclic triterpene, from now on use of a compound of the present invention, in the development of pharmaceutical and nutraceutical compositions for the prevention and/or treatment of neurodegenerative diseases, preferably sclerosis multiple.

As used herein, the term pentacyclic triterpene refers to a member of this family of compounds belonging, for illustrative purposes and without limiting the scope of the invention, to the following group: oleanolic acid, maslinic acid and erythrodiol.

A particular object of this invention is the use of a compound of the invention in which the pentacyclic triterpene comes from the oleanane family, preferably oleanolic acid.

In this invention the term “the use of a pentacyclic triterpene” also includes the use of its isomeric forms, pharmaceutically acceptable salts and solvates, synthetic derivatives such as cyano, imidazole, amide, ester and ether derivatives of parent compounds. The pentacyclic triterpenes of the present invention can be isomers, including optical isomers or enantiomers. The use of its isomers, enantiomers as well as individual diastereoisomers and mixtures thereof, are also included in the present invention. The individual diastereoisomers and mixtures thereof can be separated by conventional techniques.

Also, within the scope of this invention is the use of prodrugs of pentacyclic triterpenes. The term “prodrug”, as used herein, includes any compound derived from a pentacyclic triterpene, for example, esters, including esters of carboxylic acids, esters of amino acids, phosphate esters, sulfonate esters, metal salts, etc., Carbamates, amides, cyanide derivatives, etc. which, when administered to an individual, is capable of providing, directly or indirectly, the pentacyclic triterpene in this individual. Advantageously, the derivative is a compound that increases the bioavailability of pentacyclic triterpene when administered to an individual, or that enhances the release of same in a biological compartment. The preparation of the prodrug can be carried out by conventional methods known by those experts in the art.

As used in this invention the term “neurodegenerative disease” refers to pathologies in which cell degeneration of the central nervous system and the immuno-inflammatory component play a crucial role, and refers more specifically, for illustrative purposes and without limiting the scope of the invention, to multiple sclerosis and Alzheimer's disease.

Accordingly, another particular object of this invention is the use of a compound of the invention in the preparation of pharmaceutical or nutraceutical compositions for the prevention and/or treatment of human neurodegenerative disorders including, for illustrative purposes and without limiting the scope of the invention, multiple sclerosis and Alzheimer's disease.

Another object of this invention is a pharmaceutical composition useful for the treatment of a neurodegenerative disease, hereafter “pharmaceutical composition of this invention”, comprising a therapeutically effective amount of a pentacyclic triterpene, together with, optionally, one or more adjuvants and or a pharmaceutically acceptable vehicles.

Yet another particular object of this invention is the pharmaceutical composition of the invention in which the pentacyclic triterpene belongs to the following group: oleanolic acid, maslinic acid and erythrodiol.

A further object of this invention is the pharmaceutical composition of the invention in which the pentacyclic triterpene is oleanolic acid.

In this disclosure, the term “pharmaceutically acceptable vehicle” refers to those substances, or combination of substances, known in the pharmaceutical industry, used in the preparation of pharmaceutical forms and includes adjuvants, solids or liquids, solvents surfactants, etc.

The pharmaceutical composition may also contain one or more therapeutic agents that will eventually enhance the therapeutic action of the pentacyclic triterpene compound or increase their spectrum of activity.

The pentacyclic triterpene compound will be present in the pharmaceutical composition in a therapeutically effective amount, i.e. in an appropriate amount to exert its therapeutic effect. In a particular embodiment, the pharmaceutical composition provided by this invention, contains between 0.01% and 99.99% by weight, of a pentacyclic triterpene compound and mixtures thereof, and may be available at any appropriate dispensation form according to the administration route chosen, eg oral, parenteral, intraperitoneal or topical. A review of the different pharmaceutical forms of drug administration and preparation procedures can be found, for example, in the Treaty of Galenic Pharmacy, C. Fauli i Trillo, 1st edition, 1993, Luzán 5, SA Editions.

Yet another object of this invention is the use of the pharmaceutical composition of the invention in the treatment of a human being affected by a neurodegenerative disease, hereafter, use of the pharmaceutical composition of the present invention, consisting on the administration of the therapeutic composition which reduces the progression of the disease, preferably multiple sclerosis.

### BRIEF DESCRIPTION OF THE DRAWINGS

FIG. 1: Effect on body weight (A), and on clinical signs (B) due to the progression of the disease effected by oleanolic injected daily after first symptoms. OA1

FIG. 2: Effect on body weight (A), and on clinical signs (B) due to the progression of the disease effected by oleanolic injected daily after 7 days of immunization. OA2

FIG. 3: Oleanolic acid diminished firm arrest (A) and rolling flux (B) of leukocytes on brain microvasculature, analyzed by intravital microscopy. C, healthy mice. EAE, induced-mice. EAE+OA1, induced-mice treated with OA at the onset of the clinical signs. EAE+OA2, induced-mice treated with OA from day 7 after immunization.

FIG. 4: Changes in blood-brain barrier permeability of untreated- or OA treated EAE-mice. Evans blue dye was used as a measurement of plasma protein extravasation in (A) cerebral cortex, (B) cerebellum and (C) spinal cord 21-24 days post-immunization. C, healthy mice. EAE, induced-mice. EAE+OA1, induced-mice treated with OA at the onset of the clinical signs. EAE+OA2, induced-mice treated with OA from day 7 post-immunization.

FIG. 5: Effect of OA on osteopontin (OPN) expression in CNS tissues: (A) cerebral cortex, (B) cerebellum and (C) spinal cord, of healthy and EAE mice, analyzed by commercial ELISA kits. C, healthy mice. EAE, induced-mice. EAE+OA1, induced-mice treated with OA at the onset of the clinical signs. EAE+OA2, induced-mice treated with OA from day 7 post-immunization

FIG. 6: Survival enhancement of EAE mice due to effect of Oleanolic acid treatment, injected after first symptoms.

### BRIEF DESCRIPTION OF THE EMBODIMENTS

### Example 1

### Oleanolic Acid Reduces Clinical Signs in EAE Mice

In the experiments we used fifteen animals per group. EAE was induced as described (Slavin A. Autoimmunity 1998, 28: 109) in C57BL mice by administration of a proteolipid protein. The immunization was carried out with 100 μg of a partial peptide of myelin/oligodendrocyte glycoprotein (MOG₃₃₋₅₅) in complete Freund's adjuvant containing 4 mg of Mycobacterium tuberculosis H37Ra in 1 ml. The mice were immunized by subcutaneous injection of this emulsion on day 0. In addition, on day 0 and 2, were administered intraperitoneally 300 ng/200 μl of Bordetella pertussis toxin. The administration of 6 mg/kg of oleanolic acid was performed intraperitoneally once a day, beginning:

1.—12 days after induction of EAE, when clinical symptoms were detected, until the end of the experiment (21 days after induction) (OA1), and

2.—after 7 days of induction of EAE, before the onset of the disease, until the end of the experiment (21 days after induction) (OA2).

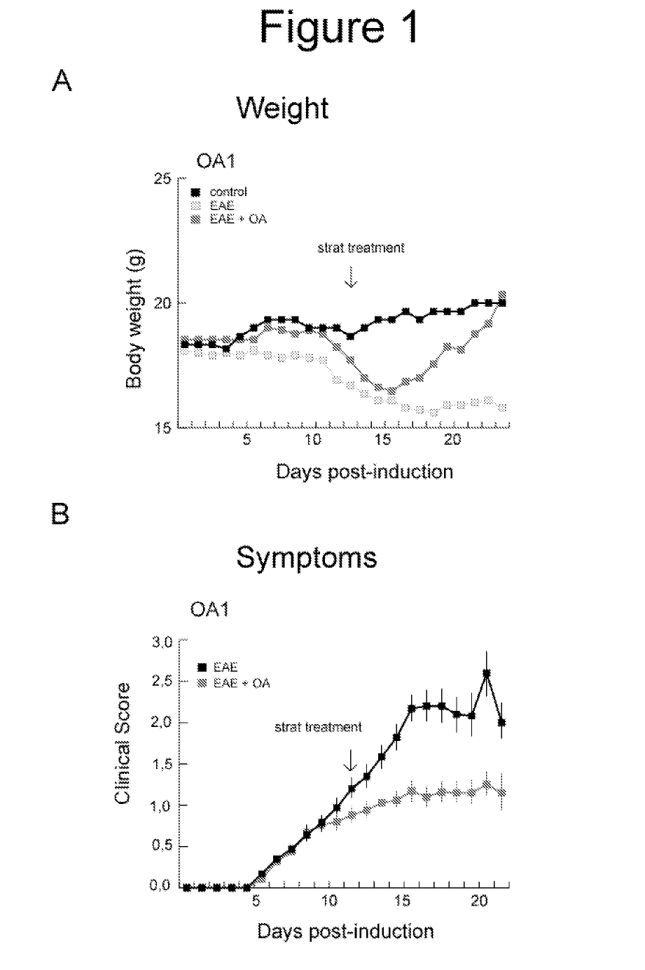

Mice were examined, weighed and scored daily in a double-blind manner for signs of EAE. OA-treated EAE mice were compared with a group of EAE animals treated with placebo, with untreated control animals or control animals received the same daily dose oleanolic acid. In addition, a pathologic evaluation was performed to confirm the effect of improving the state of the disease. The weight of the mice in each group is represented in FIGS. 1 A and 2 A. The level of paralysis reached by each group of mice is shown in FIGS. 1 B and 2 B.

### Evaluation of the Effect of Oleanolic Acid: Scores.

The effect of disease improvement (clinical effect) by the action of oleanolic acid is scored and evaluated with the pattern presented below.

Grade 0, no abnormality.

Grade 0.5, partial loss/reduced tail tone, assessed by inability to curl the distal end of the tail (Tail 50% or ⅔).

Grade 1.0, tail atony 100%

Grade 1.5, slightly/moderately clumsy gait, impaired righting ability, or combination.

Grade 2.0, hind limb weakness, partial (1 or 2)

Grade 2.5, partial (1 or 2) hind limb paralysis.

Grade 3, complete hind limb paralysis.

Grade 3.5, limb weakness.

Grade 4, tetraplegic. Complete limb paralysis

Grade 5, moribund state or death.

signs of paralysis started to develop around days 11-14 (tail atony and clumsy gait) following immunization, pointing to EAE induction;

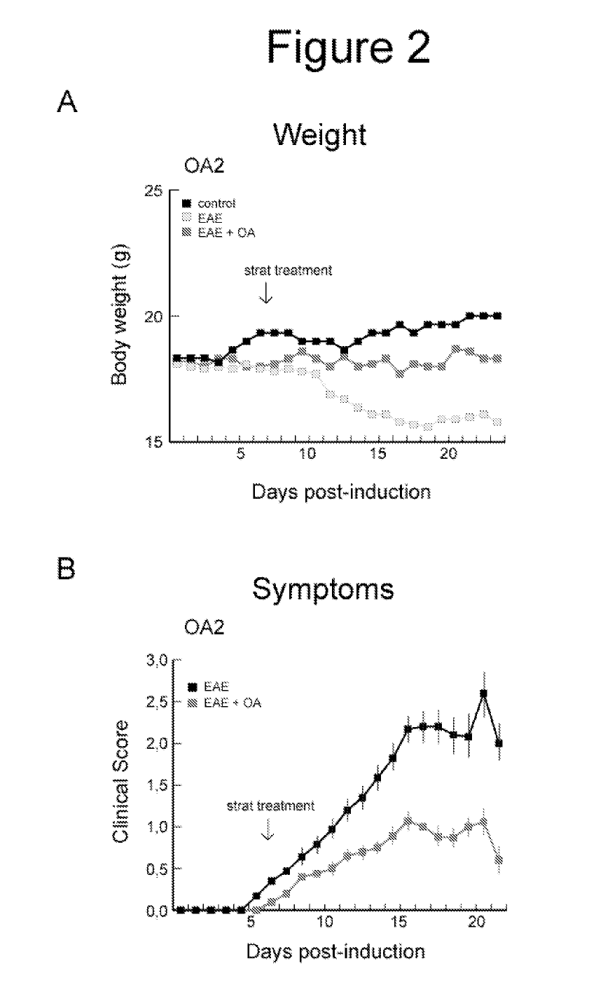

In FIG. 1B, it is shown the evolution of the signs of paralysis of untreated EAE mice, compared with OA treated EAE mice. Mice treated with 6 mg/kg of the natural triterpene compound isolated from olive pomace oil, oleanolic acid, showed a significant improvement in clinical status of the disease compared with untreated EAE group. In addition, the severity of EAE was also significantly attenuated with prophylactic administration of oleanolic acid, as there was a delay in the onset of the disease (FIG. 2B). In both situations, the reduction of body weight was suppressed, showing a recovery (FIG. 1A) or preventive (FIG. 2A) effect.

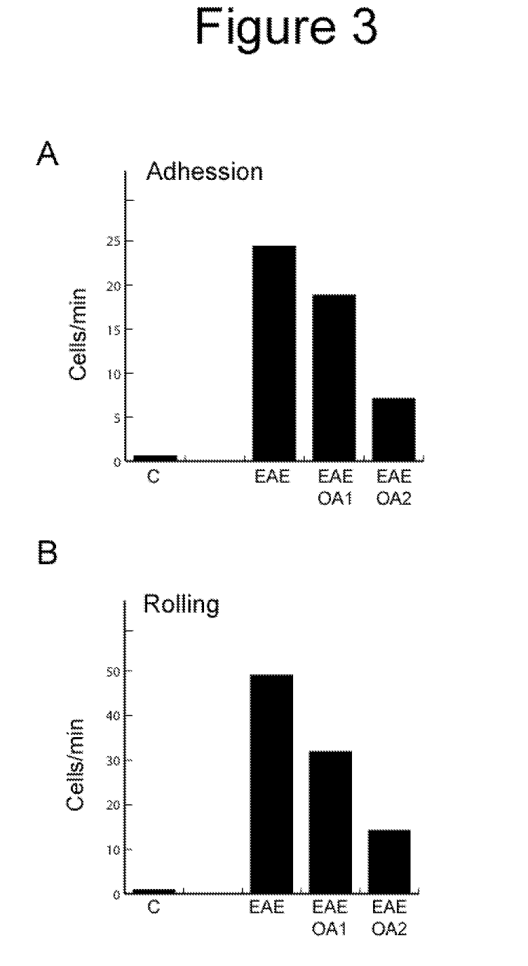

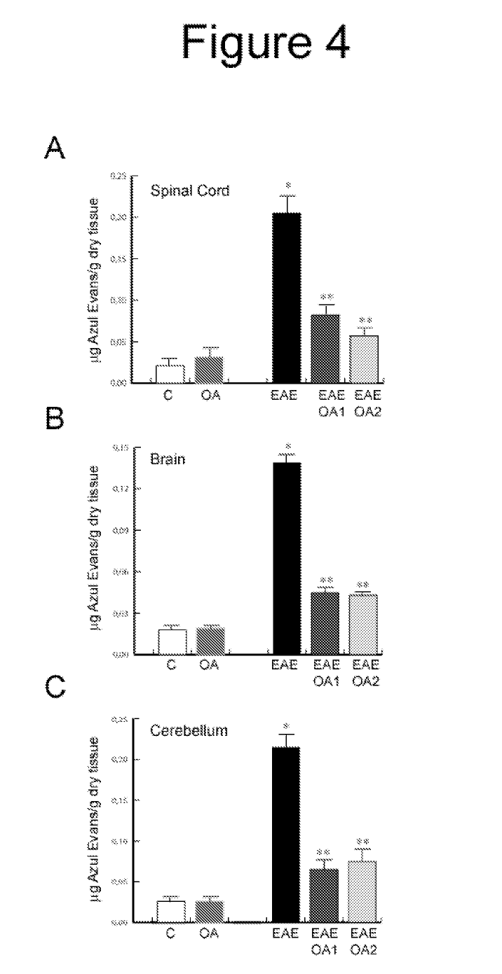

Moreover, as mentioned above, in MS there is an infiltration of inflammatory cells into the CNS because of the breakdown of the blood-brain barrier, causing both cellular and molecular extravasation. These phenomena occur mainly in the venules where blood flow is lower. To study leukocyte-endothelial interactions that occur in vivo in the microcirculation (particularly the phenomena of leukocyte rolling and adhesion), intravital fluorescence microscopy was used. FIG. 3 shows the adherent (A) and rolling (B) leukocytes. EAE animals showed a significant increase of both parameters compared to healthy controls mice. In contrast, when the mice belonged to groups of protocol EAE+OA1 or protocol EAE+OA2, we observed a significant decrease in the flow of leukocytes rolling and adhering to the endothelium, compared with the untreated EAE group. Then, to characterize the changes in vascular permeability, Evans blue was injected intraperitoneally to the different groups of animals. FIG. 4 shows the leakage that occurs in the spinal cord, brain and cerebellum. Mice of protocol EAE+OA1 and protocol EAE+OA2 have a significantly reduced leakage in the CNS tissues studied, as compared to untreated EAE animals.

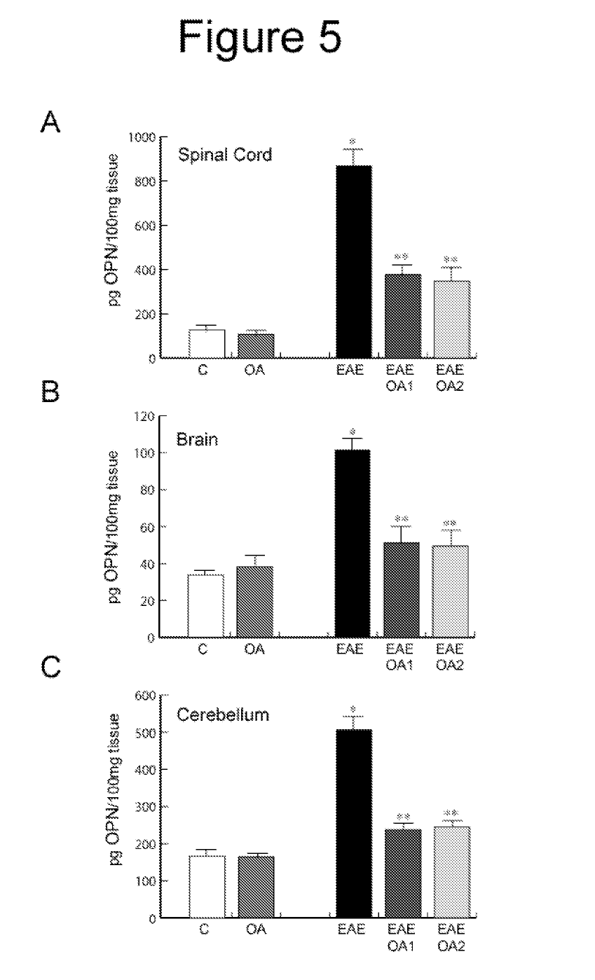

The analysis of key pro-inflammatory protein expression in EAE, lead us to study the presence and modulation of Osteopontin in CNS tissues. FIG. 5 shows that in all tissues studied, spinal cord, brain and cerebellum, the expression of this protein is increased in mice with EAE, when compared with healthy control mice. This increase was significantly reduced when mice were treated with oleanolic acid, following either protocol OA1 or OA1.

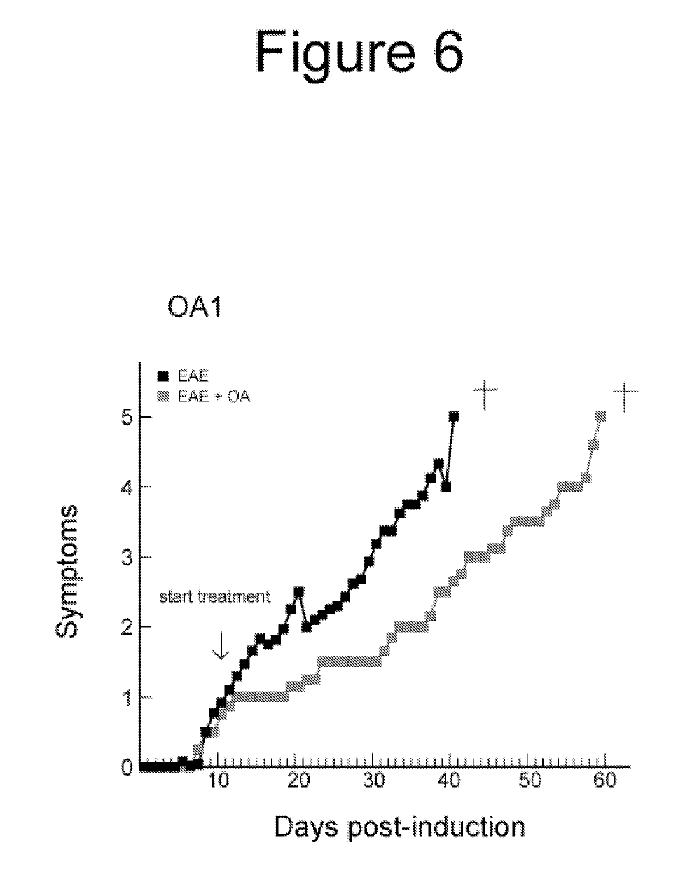

In terms of survival (FIG. 6), was noted that in the placebo-treated EAE group, mice die within the first 40 days after disease induction. In contrast, when EAE mice receive oleanolic acid at the onset of symptoms, the animals did not die until 60 days post-induction.

### Material and Methods.

### Experimental Animals.

Mice were housed in the animal care facility at the School of Medicine of the University of Valladolid. Mice were fed with a special diet for laboratory animals, water ad libitum, temperature of 20-24° C. and exposed to a light cycle of 12 h/day (8.00 am-8.00 pm) (Council of European Communities, 1986). All experimental protocols were reviewed and approved by the Animal Ethics Committee of the School of Medicine, of the University of Valladolid.

### Reagents

The following chemicals used in the experiments were provided by Sigma Chemical Co. (St. Louis, Mo., USA): Freud's complete adjuvant, M. tuberculosis H37 RA, B. pertussis toxin, rhodamine 6G and Evans Blue. The ELISA kit for detection of osteopontin was from IBL (Hamburg, Germany). The natural compound oleanolic acid was supplied by Cymit Quimica SL (Barcelona, Spain).

The pentacyclic triterpene, oleanolic acid was initially dissolved in dimethyl sulfoxide (DMSO) to prepare a stock solution of 10⁻² M. The subsequent dilutions of the triterpene were also carried out in DMSO. The final concentration of DMSO reached (less than 0.001%) did not significantly affect the results.

### Induction of EAE

Chronic progressive EAE disease was induced in adult 8-10-wk-old female C57BL/J6 mice following the protocol described by (Slavin A. Autoimmunity 1998, 28:109-20). Mice were injected in the tail base bilaterally with an innoculum containing 100 μg of MOG peptide 35-55 emulsified in complete Freund's adjuvant containing 0.4 mg/ml Mycobacterium tuberculosis H37 RA. Then, i.p. injection 300 ng/animal of B. pertussis toxin was administered by two times with an interval of 48 hours. Mice were examined daily to monitor weight loss and the onset of neurological symptoms

### Analysis of Osteopontin in the CNS.

Animals of the different experimental groups were sacrificed at day 21 post-immunization and CNS protein extracts (cerebral cortex, cerebellum and spinal cord) were prepared by homogenization in a solution: 0.4 M NaCl, 0.05% Tween 20, 0.5% BSA, 0.1 mM phenylmethylsulfonyl fluoride, 0.1 mM benzetonium chloride, 10 mM EDTA and 20 KI aprotinin (100 mg tissue/ml). The homogenate was centrifuged at 10,000 rpm for 10 min at 4° C. The concentration of osteopontin was determined in the supernatants by a commercial ELISA kit.

### Intravital Microscopy in the Brains of Mice.

Intravital microscopy techniques allow the study of leukocyte-endothelium interactions. The leukocytes that interact with the endothelial surface can be visualized because their speed is markedly reduced, as compared to the average velocity of the blood flow in the venule. To make a precise study of leukocyte-endothelium interactions, we first made a record of the venule for one minute using a camcorder, and in a second time, a more detailed analysis was performed. The parameters of interest were the number of leukocytes adhering to the venular endothelium (number of leukocytes stationary for more than 30 seconds), and the number of rolling leukocytes (number of white cells per minute moving at a velocity less than that of erythrocytes).

The craniotomy was performed on the parietal zone. Animals received i.v. administration of rhodamine 6G (0.3 mg/kg body weight) to fix leucocytes. The fluorescence associated to rhodamine 6G was visualized at 510-560 nm by epi-ilumination. The microscope (20×) was used for observing micro-circulating events in brain. A digital camera coupled with a microscope displays the images on a monitor, which were recorded on video for further analysis of the leucocyte in rolling and adhesion.

### Analysis of Extravasation Alterations

Evans blue dye is able to bind quantitatively to albumin, both in vivo and in vitro. This property has been widely used for quantification of protein extravasation as an index of increased vascular permeability and, indirectly, of tissue damage. The Evans Blue, once extravasated into the tissues, is removed from them, and then quantified by visible spectrophotometry.

Mice from all experimental groups were given 30 mg/kg of Evans Blue i.p. Then, the spinal cord, cerebrum and cerebellum was removed and washed in saline. The dye was extracted from the CNS tissues with formamide and the concentration was determined by measuring the absorvance at 620 nm. CNS tissue was dried 24 h at 60° C. and weighed. The extravasated dye was expressed as μg of Evans Blue/mg dried weight of the tissue.

### Statistical Analyses

Data were treated using Mann-Whitney U test. Results are expressed as mean±SD; P values <0.05 were considered statistically significant.

1. A method for treating a neurogenerative disease in a patient in need thereof, which comprises:
administering to the patient an effective amount of a composition comprising a pentacyclic triterpene.

2. The method of claim 1, wherein the pentacyclic triterpene is selected from among oleanolic acid, maslinic acid and erythrodiol.

3. The method of claim 1, wherein the pentacyclic triterpene is oleanolic acid (3β-hydroxyolean-12-en-28-oic acid).

4. A pharmaceutical composition for the treatment of neurodegenerative diseases wherein the composition comprises a therapeutically effective amount of a pentacyclic triterpene, together with, optionally, one or more adjuvants and/or pharmaceutically acceptable vehicles.

5. The pharmaceutical composition according to claim 4 wherein the pentacyclic triterpene is selected from among oleanolic acid, maslinic acid and erythrodiol.

6. The pharmaceutical composition according to claim 4 wherein the pentacyclic triterpene is oleanolic acid (3β-hydroxyolean-12-en-28-oic acid).

7. A method for treating a neurogenerative disease in a patient in need thereof, which comprises:
administering to the patient the composition of claim 4.

8. The method of claim 1, wherein the neurogenerative disease is multiple sclerosis.

9. The method of claim 7, wherein the neurogenerative disease is multiple sclerosis.
